(19) United States
(12) Patent Application Publication
(10) Pub. No.: US 2025/0155417 A1
(43) Pub. Date: May 15, 2025

(54) SYSTEMS AND METHODS FOR MONITORING EMISSIONS FROM LIVESTOCK

(71) Applicant: C-LOCK, INC., Rapid City, SD (US)

(72) Inventors: ROBERT SCOTT ZIMMERMAN, Rapid City, SD (US); PATRICK R. ZIMMERMAN, Rapid City, SD (US)

(73) Assignee: C-LOCK, INC., Rapid City, SD (US)

(21) Appl. No.: 18/941,396

(22) Filed: Nov. 8, 2024

Related U.S. Application Data
(60) Provisional application No. 63/548,351, filed on Nov. 13, 2023.

(51) Int. Cl. G01N 33/00; G01N 33/497

(52) U.S. Cl. CPC G01N 33/0047; G01N 33/004; G01N 33/4975

(57) ABSTRACT

An emissions measurement system to measure emissions from an animal is provided comprising a fixture in fluid communication with an air handler, wherein the air handler comprises a blower and a first emissions sensor.

### CROSS-REFERENCE TO RELATED APPLICATIONS

This application is a non-provisional of, and claims priority to U.S. Provisional Application No. 63/548,351, filed Nov. 13, 2023, entitled “SYSTEMS AND METHODS FOR MONITORING EMISSIONS FROM LIVESTOCK,” which is herein incorporated by reference in its entirety.

### FIELD

The present disclosure relates to systems and methods for monitoring emissions from livestock.

### BACKGROUND

Metabolic gas emissions from cattle and other ruminant animals can provide important information relative to the animal's health, feed efficiency, and metabolic state. Metabolic gasses, such as carbon dioxide and methane, have been found to be correlated with feed intake. Further, methane is produced in large quantities during the ruminant animal's digestion process. It is primarily emitted from the muzzle of the animal once every 40-60 seconds, similar to a belch in humans. Methane is a potent greenhouse gas that contributes to global climate change, and therefore, cattle and other ruminant animals are an important source of global greenhouse gas emissions. There is a focus on using different management strategies to reduce methane emissions. Further, other gasses, like carbon dioxide can be highly correlated with the animal's feed intake and therefore can be useful as a proxy measure for feed intake.

Breed and genetic improvements are key strategies to produce more feed-efficient and lower methane-emitting animals. This uses measurement methods to determine the amount of feed individual animals consume and the amount of methane they emit to identify the most feed-efficient and lowest methane-emitting animals. Determining which genetics influence emissions requires measuring large numbers of animals, sometimes thousands.

Metabolic gas measurement technologies aid in developing and testing supplements and pharmaceutical drugs that reduce methane and improve ruminant efficiency. Further, once mitigation strategies are undertaken, either to reduce greenhouse gas emissions from ruminants or to improve feed efficiency, there will be a need to allow quick measurement within the farm environments to determine the effectiveness of the mitigation strategies. Periodic auditing will be needed if carbon credits are generated for specific mitigation practices.

### SUMMARY

In various embodiments, an emissions measurement system is provided comprising a fixture in fluid communication with an air handler, wherein the air handler comprises a blower and a first emissions sensor.

In various embodiments, a method is provided comprising affixing a fixture at least partially around a muzzle of a ruminant, creating an airflow through the fixture and into a tube, receiving the airflow at an air handler, measuring at least one of a methane concentration or a carbon dioxide concentration in the airflow.

In various embodiments, an emissions measurement system is provided comprising a fixture comprising a mounting feature configured to couple to a nose clip, wherein the fixture is configured to at least partially surround a muzzle of an animal and to at least partially define an air gap between the fixture and the muzzle.

The present disclosure may include any one or more of the individual features disclosed above and/or below alone or in any combination thereof. The foregoing features and elements may be combined in various combinations without exclusivity, unless expressly indicated herein otherwise. These features and elements as well as the operation of the disclosed embodiments will become more apparent in light of the following description and accompanying drawings.

### BRIEF DESCRIPTION OF THE DRAWINGS

The subject matter of the present disclosure is particularly pointed out and distinctly claimed in the concluding portion of the specification. A more complete understanding of the present disclosure, however, may best be obtained by referring to the detailed description and claims when considered in connection with the drawing figures, wherein like numerals denote like elements.

FIG. 1 illustrates a system for measuring emissions in livestock, in accordance with various embodiments.

FIG. 2 illustrates a system for measuring emissions in livestock, in accordance with various embodiments.

FIG. 3 illustrates a nose clip, in accordance with various embodiments.

FIG. 4 illustrates a cross section of a nose clip and fixture, in accordance with various embodiments.

FIG. 5 illustrates a nose clip, in accordance with various embodiments.

FIG. 6 illustrates carbon dioxide emissions over time, in accordance with various embodiments.

FIG. 7 illustrates methane emissions over time, in accordance with various embodiments.

### DETAILED DESCRIPTION

The detailed description of embodiments herein makes reference to the accompanying drawings, which show embodiments by way of illustration. While these embodiments are described in sufficient detail to enable those skilled in the art to practice the disclosure, it should be understood that other embodiments may be realized and that logical, chemical, and mechanical changes may be made without departing from the spirit and scope of the disclosure. Thus, the detailed description herein is presented for purposes of illustration only and not for limitation. For example, any reference to singular includes plural embodiments, and any reference to more than one component or step may include a singular embodiment or step. Also, any reference to attached, fixed, connected or the like may include permanent, removable, temporary, partial, full and/or any other possible attachment option. Further, any steps in a method discussed herein may be performed in any suitable order or combination. Additionally, any reference to without contact (or similar phrases) may also include reduced contact or minimal contact. It should also be understood that unless specifically stated otherwise, references to “a,” “an,” or “the” may include one or more than one and that reference to an item in the singular may also include the item in the plural.

Further, all ranges may include upper and lower values and all ranges and ratio limits disclosed herein may be combined.

As mentioned above, animals, in particular bovines, are not often suited to having devices placed on or tightly around the muzzle. First, the bovine may become stressed or agitated, which is not healthy for the bovine and/or dangerous for animal handlers. Taking time to train each bovine to be measured is time consuming, expensive, and not always successful for each animal. Second, a tightly affixed mask on a bovine may risk suffocation of the bovine if airflow is not sufficient. Both these obstacles, among others, tend to prevent the quick and easy measurement of emissions from large numbers of animals. It should be noted that while various embodiments are particularly suited to ruminants, such as bovines, various embodiments of the present disclosure are suitable for use with porcine animals, sheep, goats, and other livestock.

In various embodiments, an emissions measurement system is provided. In various embodiments, the emissions measurement system may quickly and quantitatively measure mass flux rates of metabolic gasses from cattle, including emissions of methane, carbon dioxide, hydrogen, and oxygen absorption, among other emissions. In various embodiments, high airflow rates are used to allow for a loose fitting fixture that entrains ambient air around the mouth and/or nose and/or muzzle of the animal. By using high air flow rates, the risk of suffocation is at least one of reduced or eliminated. Moreover, a loose fitting fixture is typically readily accepted by an animal without training or other preparation work. Further, by using high airflow rates, the time needed to generate useable data is lessened, thus leading to a shorter sample time per animal. In various embodiments, attachment to the animal via a nose ring simplifies attachment to the animal and is better tolerated/is more comfortable for the animal than the use of straps.

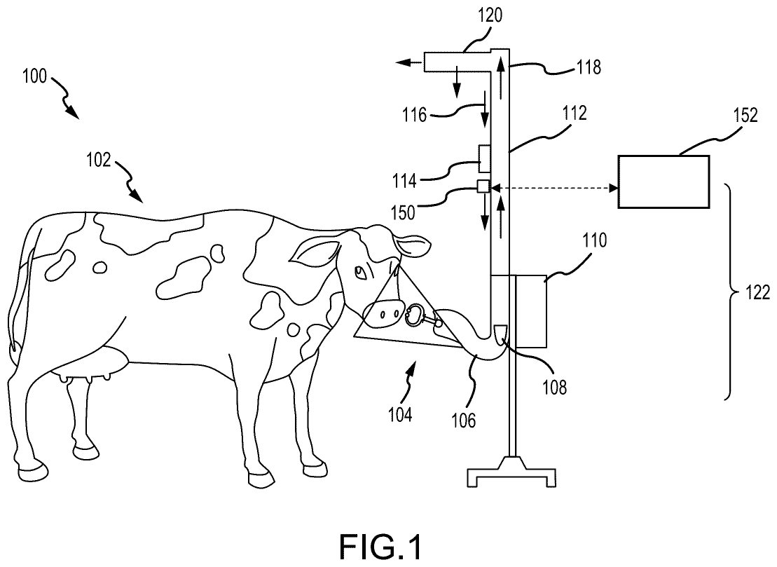

With reference to FIG. 1, an emission measurement system 100 is disclosed. A fixture 104 is shown coupled to an animal (here, a bovine) 102. Fixture 104 is in fluid communication with air handler 122 via tube 106. Air handler 122 is in fluid communication with pipe 112, pipe 112 having an exhaust outlet 116. Fan 118, also referred to as blower 118, is disposed in pipe 112 and is in fluid communication with the same. Airflow meter 114 is disposed at least partially in pipe 112 and in fluid communication with the same.

Fixture 104 comprises a structure configured to fit around the nares and mouth (together, also referred to as a muzzle) of an animal, such as bovine 102. Fixture 104 may comprise a rigid or semi-rigid structure in any suitable configuration. In various embodiments, fixture 104 comprises a cylindrical structure having a base and an opening to fit at least partially around the muzzle of an animal. The fixture 104 may comprise at least one of a rigid plastic, metal, or composite materials such as carbon fiber, fiberglass, garolite (i.e., G10 composite material). Where fixture 104 comprises a rigid structure, the fixture 104 may comprise an opening (aperture) to be placed over the muzzle, forming an airgap between the muzzle and the fixture 104. In various embodiments, fixture 104 is constructed of a textile material such as nylon, ballistic nylon, polyester, carbon fiber, cotton, hemp, polycarbonate, thermoplastics, rubber, synthetic rubber, silicone, and other suitable textiles. In embodiments where fixture 104 is constructed of a textile, in various embodiments, a rigid member may support the area of the fixture 104 that fits around the muzzle of the animal. In that regard, a hoop, such as a collapsible hoop, or other structure may rigidly support the textile. In such embodiments, the hoop or other rigid member may form an airgap around the muzzle of the animal.

In various embodiments, the airgap of the fixture 104 about the muzzle of the animal 102 allows ambient air to be in fluid communication with the inside of the fixture 104.

Fixture 104 comprises a mounting feature configured to be coupled to a nose clip. The mounting feature may be located on the base of the fixture 104. The mounting feature may comprise a clip, fastener, male or female threaded coupling, carabiner, nylon webbing, hook, or other suitable coupling. The mounting feature may allow for removable coupling of the fixture 104. A nose clip may be fit into the nares and/or nostrils of the muzzle of the animal while coupled to the fixture 104. In that regard, the nose clip may act to retain the fixture to the muzzle of the animal without causing pain or discomfort to the animal, as further illustrated in detail below.

The base of fixture 104 may comprise an aperture or other passageway. In that regard, the fixture 104 is placed in fluid communication with tube 106. Tube 106 may be a pipe of any suitable diameter. Tube 106 may be coupled to the fixture 104 in an airtight or substantially airtight manner. Thus, little to no air is allowed to escape fixture 104 where fixture 104 joins tube 106.

Air handler 122 is in fluid communication with fixture 104 via tube 106. Air handler 122 receives airflow from tube 106 and has one or more emissions sensors disposed at least partially in the airflow. Emissions sensors may be configured to measure emissions concentration in the airflow such concentrations of carbon dioxide, hydrogen, methane, organic compounds, oxygen, and any other material in gaseous or vapor form emitted from an animal's muzzle. In various embodiments, emissions sensors may detect the presence of various organic and in organic compounds emanating from the muzzle of an animal. For example, detection of ammonia and/or acetone in an emissions stream from an animal may be useful in assessing emission output as well as to address the health of the animal. Moreover, in various embodiments, emission sensors may be configured to detect markers indicative of infectious disease, such as the presence of bacteria, fungus, or viruses. In that regard, emissions sensors may be configured to detect viral, bacterial, or fungal DNA, RNA, and/or proteins and/or fragments of the foregoing.

Emission sensors that measure methane and carbon dioxide can be measured with a non-dispersive infrared, tunable diode laser, or electrochemical sensors, while hydrogen can be measured using electrochemical sensors, and oxygen concentrations can be measured by paramagnetic, electrochemical, or luminescence sensors. Non-dispersive infrared sensors and tunable diodes may be configured as emissions sensors to detect acetone. Photoionization detectors, flame ionization detectors, and metal oxide semiconductor sensors may be used to measure the concentrations of various volatile organic compounds.

Portions of the airflow may be continuously or periodically withdrawn from the airflow and routed into the concentration sensors or, in various embodiments, the sensors may be disposed in the airflow itself. In various embodiments, at periodic intervals, portions of the emissions stream may be routed to various emission sensors for measurements. In various embodiments, however, emissions sensors are at least partially disposed in the emissions stream. Both periodic and continuous sampling may be used concurrently. For example, methane may be measured directly from the emission stream while a periodic sub sample may be taken for detection of biological infectious agents. In various embodiments, the emissions sensors log high-resolution data every second or multiple times per second, allowing the air handler 122 to record the methane and carbon dioxide concentration peaks, among other data, that occur from each eructation (roughly every 40-60 seconds) and respirated gasses.

Air handler 122 further comprises a processor 150 and may comprise an airflow meter 114. In various embodiments, airflow meter 114 is disposed on pipe 112 and is in electronic and/or logical communication with air handler 122, specifically with processor 150 of airflow handler 114. Airflow meter 114 may comprise a hot-film or hot-wire anemometer, or pressure differential sensor to measure airflow accurately. Airflow rate from airflow meter 114 may be communicated to the processor 150 of air handler 122.

Blower 118 comprises a device capable of moving air through air handler 122, tube 106, and fixture 104. Blower 118 may comprise any suitable device, including a fan and/or impeller driven by a motor, such as a brushless DC motor, to cause air to flow through pipe 112. Blower 118 is capable of high flow rates. In various embodiments, blower 118 may cause airflow rates of between 150 liters per minute to 2500 liters per minute, 200 liters per minute to 2000 liters per minute, 250 liters per minute to 1000 liters per minute, 250 liters per minute and/or 1800 liters per minute. By using high flow rates (above 250 L/min), combined with the air gap formed between fixture 104 and the muzzle of the animal, airflow around the animal's muzzle will be sufficient for the animal to respirate (breath) while being able to capture gaseous emissions. The high flow rate thus reduces or eliminates the risk of suffocation while allowing for a loose-fitting fixture 104 that minimizes or removes the need to train an animal to use fixture 104. In contrast, airtight hoods or other devices that affix to an animal's muzzle must be closely monitored as a power failure or other equipment failure may lead to suffocation of the animal unless action is taken swiftly. In various embodiments, the air gap formed between the fixture 104 and the animals' muzzle allows the animal to continue breathing safely in the event of equipment failure, malfunction, or a power outage.

The mass flux rate of the metabolic gasses at any given time can be determined by multiplying the airflow rate by the increase or decrease in concentration of each gas from its background and using ideal gas laws to convert gas volume to gas mass. To calculate average flux for the total time of each animal measurement, all the gas flows are averaged, such as for 3-7 minutes per animal. This means that using systems in accordance with the present disclosure may be used to sample a large number of animals in a given day, providing researchers with a sufficiently high sample size of data that may be useful for research and management purposes.

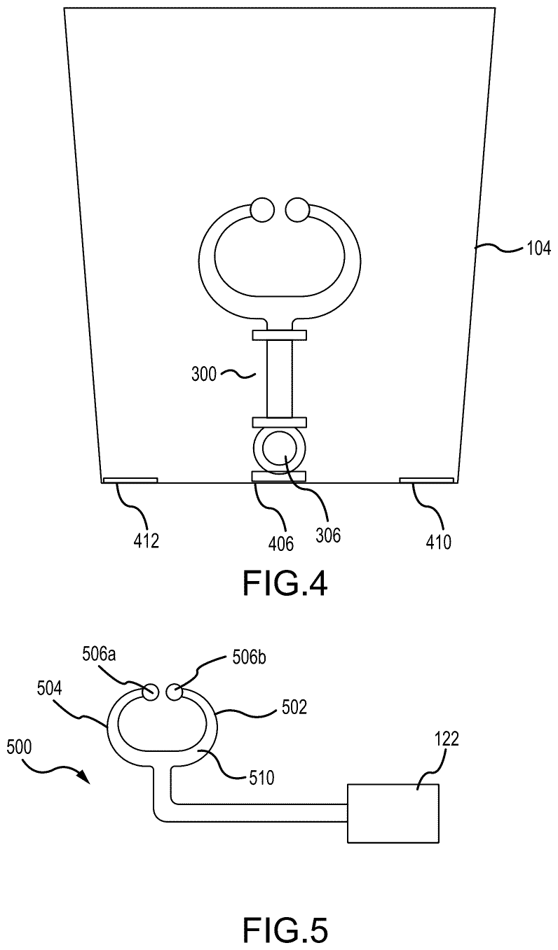

In various embodiments, an acceleration or head sensor (e.g., accelerometer 410, with reference to FIG. 4) may be used in emission measurement system 100 to generate head movement data to determine if, and/or how often, the animal is shaking its head, which can cause sample loss. Accelerometer 410 may be in electronic communication with processor 150, whether in direct wired connection or via a wireless protocol. Accelerometer 410 may convey the head movement data to processor 150 so processor 150 may be used to filter out time periods when the animal is not still or substantially still. In various embodiments, processor 150 may forward the raw head movement data to a cloud computing infrastructure 152 and the cloud computing infrastructure 152 may perform the filtering. In this manner, metadata relating to identifying the head movement and the associated emissions data that were collected during the head movement may be identified so that the associated emissions data may be filtered by the cloud computing infrastructure 152 in the event such filtering is appropriate.

Additional sensors 412 may be coupled to fixture 104 in any suitable location. Additional sensors 412 may comprise one or more sensors in electronic communication with the processor 150. Additional sensors 412 may comprise an infrared temperature sensor, a microphone, and a thermometer. In embodiments where additional sensors 412 includes an infrared temperature sensor, the infrared temperature sensor may be used to measure the eye temperature of the animal and the measurement delivered to processor 150 and/or cloud computing infrastructure 152. The eye temperature may then be associated with an internal body temperature of the animal. In embodiments where additional sensors 412 includes a microphone, audio may be captured and sent to processor 150 and/or cloud computing infrastructure 152 for storage and/or analysis. Audio may be assessed for the presence of wheezing or other health indicator. In embodiments where additional sensors 412 includes a thermometer, the temperature of the gaseous emissions and/or ambient temperature may be measured and recorded by processor 150 and/or cloud computing infrastructure 152.

In various embodiments, emission measurement system 100 may include an electronic radio frequency identification system to automatically identify and log animals with electronic tags. In that regard, processor 150 may be configured to receive animal identification data from an electronic radio frequency identification system associated with an animal. Radio frequency identification tags may also be placed in fixture 104. In this way, a user may identify and log the radio frequency identification tags associated with the animal and the radio frequency identification associated with the fixture at similar times or at the same time. The radio frequency identification tag data associated with the animal and the radio frequency identification tag data associated with the emission measurement system 100 may be automatically linked. When using multiple emission measurement systems 100, a user may then later reference the specific animal data with the specific emission measurement system 100 data.

In various embodiments, emission measurement system 100, and in particular processor 150, communicates with a cellular telephone network, cloud computing infrastructure 152, or computer application, so that emissions measurements and/or other relevant data (i.e., metadata relating to the emissions data) about the animal can be entered, including but not limited to, farm identification number, animal body weight, breed, milk production data, location measured, age, and/or dietary data.

In various embodiments, emission measurement system 100 includes a method to transmit data wirelessly to cloud computing infrastructure 152 for further remote analysis. For example, emission measurement system 100 may use a ethernet wire, wireless communication protocol (e.g., Bluetooth, Bluetooth LE, NFC, TCP/IP, Wi-Fi, etc.) or a cellular modem to transmit data to cloud computing infrastructure 152. Cloud computing infrastructure 152 may store emissions data and metadata related thereto and may provide data analysis to produce actionable data for herd management, heard health care, emissions management, and other purposes to further the health and safety of the animals measured as well as to improve operational efficiency.

In various embodiments, air handler 122 may house blower 118 and air flow meter 114, improving portability.

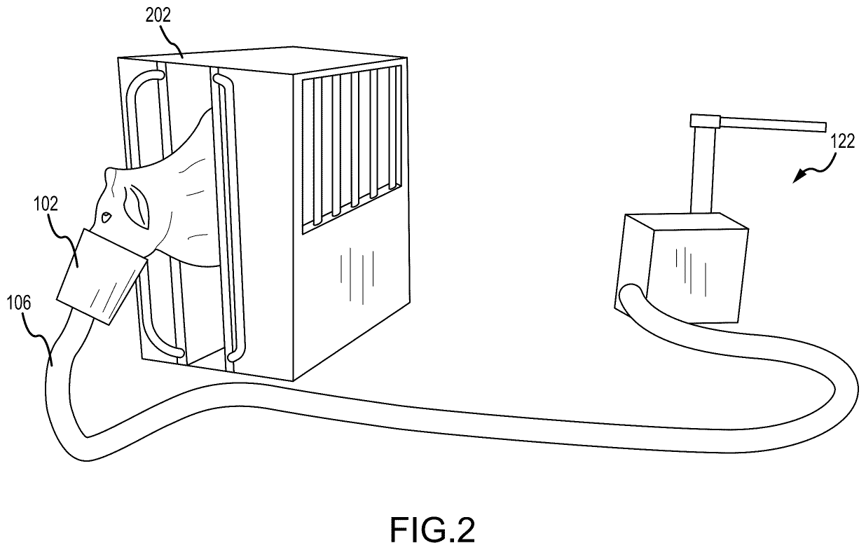

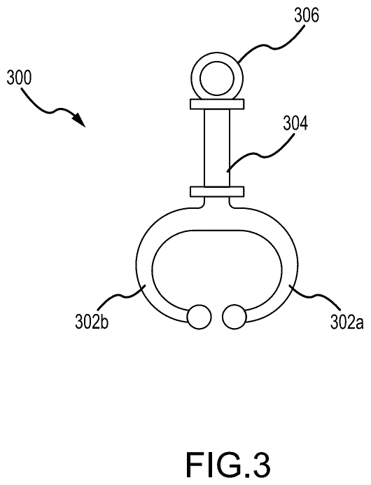

With reference to FIG. 2, animal 102 is held in squeeze chute 202. In various embodiments, a headlock, such as that often used in dairy operations, may be used in place of squeeze chute 202. Fixture 104 is affixed to animal's 102 muzzle via a nose clip 300 (with momentary reference to FIG. 3). Air handler 122, which here holds blower 118, produces a high airflow rate through tube 106. After minutes of sample time (3-10 minutes), sufficient data is collected and the next animal may be sampled.

With reference to FIG. 3, nose clip 300 is illustrated. Nose clip 300 comprises coupling fitting 306. Coupling fitting 306 is illustrated as a loop, but may also comprise a carabiner, a threaded coupling, a hook, a clasp, and/or other suitable structures for removable coupling to fixture 104. Shaft 304 connects coupling fitting 306 to arc portions 302a and 302b. Arc portions 302a and 302b comprise a smooth surface so as to be inserted into the nares and/or nostrils of an animal, capable of supporting fixture 104 comfortably and without pain.

With reference to FIG. 4, nose clip 300 is illustrated coupled to fixture 104. Coupling fitting 306 is coupled to mounting feature 406. Mounting feature 406 may comprise a carabiner, nylon webbing, a threaded coupling, a hook, a clasp, and/or other suitable structures for removable coupling to nose clip 300. In various embodiments, mounting feature 406 may comprise a clamping assembly in which a rope may be pulled through a ratchet mechanism (e.g., ratchet and pawl mechanism) to tighten the fixture 104 around the animal's muzzle. A button within the fixture 104 can be configured to release the tension from the rope, thereby releasing the rope from the clamping assembly and allowing removal of the fixture 104. In that regard, mounting feature 406 and coupling fitting 306 mate to provide retention of fixture 104 to the animal's muzzle via nose clip 300.

With reference to FIG. 5, system 500 is illustrated. In system 500, fixture 104 is not needed because an air passageway is provided directly via nose clip 510. Nose clip 510 has arc portions 504 and 502 to affix to the nares and/or nostrils of an animal. Nose clip 510 also comprises air passageways 506a and 506b. Air passageways 506a and 506b may be embedded within arc portions 504 and 502 such that arc portions 504 and 502 comprise apertures that allow air from the nares and/or nostril of the animal to be taken through air passageways 506a and 506b, though in various embodiments, air passageways 506a and 506b may comprise tubes affixed to the outside of arc portions 504 and 502. Air handler 122 is fluidly coupled to air passageways 506a and 506b and thus may cause an airflow to occur within nose clip 510 so as to entrain gas escaping from the nares and/or nostrils of an animal as well as entrained ambient air. In system 500, given the relatively smaller cross sections of art portions 502 and 504, sufficient air flow may not be able to be generated to determine flux. Air handler 122 of system 500 may determine concentration only measurements of emissions. In that regard, system 500 allows for an even more compact solution that allows ease of use and an even higher rate of animal acceptance. It should be understood that the emissions captured in system 500 include both the tidal breath of the animal (i.e., the typical exhalation of air from the lungs) as well as eructations (i.e., the gaseous emissions from the animal's gut).

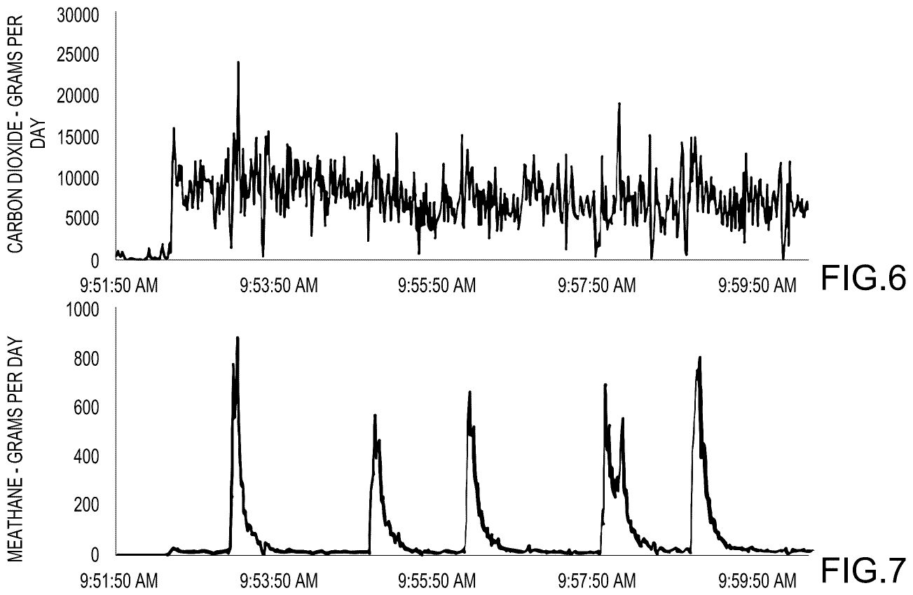

FIG. 6 shows a sample of output of carbon dioxide over time and FIG. 7 shows a sample output of methane over time. The output illustrates the periodic emissions of these gases in the overall breathing of an animal. By taking a high resolution sample (i.e., many measurements over a small amount of time, such as 1-10 measurements per second), emissions may be confidently sampled over a large number of animals. The high resolution sampling also allows for data to show each erucation. In that regard, the number of erucations per minute or hour per animal may be determined. Moreover, due to the short measurement times, animals may be measured multiple times per day to capture effects of feed schedule, circadian rhythms, and other factors that affect emissions throughout the course of a day.

Sampling can be repeated over time (for example once per day, week, or month) to determine trends and changes in emissions over time. Increasing the numbers of samples will reduce the uncertainty when averaging a more significant number of samples. Systems in accordance with this disclosure could be used to measure the mass flow rate of animal emissions resulting from changes in diet, intake, or dietary supplements. Thus, such systems could be used as a quick and easy way to determine emissions inventories, audit the effects of supplementation or measure emissions phenotypes from animals.

Air handler 122 may transmit measurement data, such as that shown in FIGS. 6 and 7, to a cloud computing infrastructure 152 or store such data locally for later upload to a cloud computing infrastructure 152. The cloud computer infrastructure may then collect, house, and analyze emissions data across one or more facilities and herds, thus being able to quantify emissions of an organization and to reveal how changes in herd management techniques affect overall emissions.

Benefits and other advantages have been described herein with regard to specific embodiments. Furthermore, the connecting lines shown in the various figures contained herein are intended to represent exemplary functional relationships and/or physical couplings between the various elements. It should be noted that many alternative or additional functional relationships or physical connections may be present in a practical system. However, the benefits, advantages, solutions to problems, and any elements that may cause any benefit, advantage, or solution to occur or become more pronounced are not to be construed as critical, required, or essential features or elements of the disclosure. The scope of the disclosure is accordingly to be limited by nothing other than the appended claims, in which reference to an element in the singular is not intended to mean “one and only one” unless explicitly so stated, but rather “one or more.” Moreover, where a phrase similar to “at least one of A, B, or C” is used in the claims, it is intended that the phrase be interpreted to mean that A alone may be present in an embodiment, B alone may be present in an embodiment, C alone may be present in an embodiment, or that any combination of the elements A, B and C may be present in a single embodiment; for example, A and B, A and C, B and C, or A and B and C.

Systems, methods, and apparatus are provided herein. In the detailed description herein, references to “one embodiment,” “an embodiment,” “an example embodiment,” etc., indicate that the embodiment described may include a particular feature, structure, or characteristic, but every embodiment may not necessarily include the particular feature, structure, or characteristic. Moreover, such phrases are not necessarily referring to the same embodiment. Further, when a particular feature, structure, or characteristic is described in connection with an embodiment, it is submitted that it is within the knowledge of one skilled in the art to affect such feature, structure, or characteristic in connection with other embodiments whether or not explicitly described. After reading the description, it will be apparent to one skilled in the relevant art(s) how to implement the disclosure in alternative embodiments.

Furthermore, no element, component, or method step in the present disclosure is intended to be dedicated to the public regardless of whether the element, component, or method step is explicitly recited in the claims. No claim element herein is intended to invoke 35 U.S.C. 112 (f) unless the element is expressly recited using the phrase “means for.” As used herein, the terms “comprises,” “comprising,” or any other variation thereof, are intended to cover a non-exclusive inclusion, such that a process, method, article, or apparatus that comprises a list of elements does not include only those elements but may include other elements not expressly listed or inherent to such process, method, article, or apparatus.

What is claimed is:

1. An emissions measurement system comprising:
a fixture in fluid communication with an air handler;
wherein the air handler comprises a blower and a first emissions sensor.

2. The emissions measurement system of claim 1, wherein the air blower is capable of generating an airflow of at least 250 L/minute.

3. The emissions measurement system of claim 2, wherein the first emissions sensor senses a concentration of methane in the airflow.

4. The emissions measurement system of claim 3, wherein the fixture comprises a mounting feature configured to couple to a nose clip.

5. The emissions measurement system of claim 4, wherein the fixture comprises an aperture in fluid communication with a tube, the tube in fluid communication with the air handler and the first emissions sensor.

6. The emissions measurement system of claim 5, wherein the air handler comprises a second emissions sensor, wherein the second emissions sensor senses a concentration of carbon dioxide in the airflow.

7. The emissions measurement system of claim 6, wherein the air handler comprises an air filter disposed in the airflow and an airflow meter disposed in the airflow.

8. The emissions measurement system of claim 7, wherein the air handler comprises a processor in electronic communication with the first emissions sensor, second emissions sensor, and blower.

9. The emissions measurement system of claim 8, wherein the processor is configured to receive a methane concentration of the airflow from the first emissions sensor and at least one of store the methane concentration in a local memory or transmit the methane concentration to a cloud computing system.

10. The emissions measurement system of claim 9, wherein the processor is configured to receive a carbon dioxide concentration from the second emissions sensor and at least one of store the carbon dioxide concentration in the local memory or transmit the carbon dioxide concentration to the cloud computing system.

11. The emissions measurement system of claim 7, wherein the fixture comprises a cylindrical structure having a base, the base having an aperture coupled to the tube.

12. The emissions measurement system of claim 11, wherein the fixture is configured to at least partially surround a muzzle of a ruminant.

13. The emissions measurement system of claim 12, wherein, in response to the fixture being affixed to the muzzle of the ruminant, an airgap is formed at least partially around the muzzle.

14. The emissions measurement system of claim 13, wherein, in response to the fixture being affixed to the muzzle of the ruminant, an airgap is formed at least partially around the muzzle.

15. The emissions measurement system of claim 14, wherein the fixture comprises at least one of a rigid plastic or a textile.

16. The emissions measurement system of claim 8, further comprising at least one of a head movement sensor or accelerometer to gather head movement data, wherein the processor is configured to identify head movement data.

17. The emissions measurement system of claim 16, wherein the processor is configured to receive an electronic animal identification to identify the animal using an electronic tag, wherein the processor is configured to be in electronic communication with a second processor configured to receive metadata including animal identification and biological parameters, and wherein the processor is configured to wirelessly transmit data to a cloud computing infrastructure.

18. A method comprising:
affixing a fixture at least partially around a muzzle of a ruminant;
creating an airflow through the fixture and into a tube;
receiving the airflow at an air handler;
measuring at least one of a methane concentration or a carbon dioxide concentration in the airflow.

19. A nose clip comprising a first curved portion and a second curved portion, the first curved portion coupled to the second curved portion at a pivot; the first curved portion having a first tube coupled thereto, the second curved portion having a second tube coupled thereto.

20. The nose clip of claim 19, wherein the first tube and the second tube are fluidly coupled.
